(19) United States
(12) Patent Application Publication
(10) Pub. No.: US 2024/0344992 A1
(43) Pub. Date: Oct. 17, 2024

(54) SYSTEMS AND METHODS FOR PREDICTING SOIL CARBON CONTENT

(71) Applicant: TERRAMERA, INC., Vancouver (CA)

(72) Inventors: Austin Caulfield ROTHWELL, Vancouver (CA); Travis Godwin GOOD, Vancouver (CA); Hoda AGHAEI, Vancouver (CA); Maryamsadat RASOULIDANESH, Vancouver (CA); Sadegh SHOKATIAN, Vancouver (CA); Vinod PARMAR, Vancouver (CA); Conor Andrew SEXTON, Vancouver (CA)

(21) Appl. No.: 18/731,661

(22) Filed: Jun. 3, 2024

Related U.S. Application Data
(63) Continuation of application No. PCT/CA2022/051760, filed on Dec. 1, 2022.
(60) Provisional application No. 63/285,942, filed on Dec. 3, 2021.

(51) Int. Cl. G01N 21/65; G01N 33/24

(52) U.S. Cl. CPC G01N 21/65; G01N 33/24

(57) ABSTRACT

Machine learning models for generating predictions of sequestered soil carbon content are provided. The models can be trained on synthetic training data with associated synthetic spectral measurements and ground truth training data with associated ground truth spectral measurements. Systems and methods for training such machine learning models are also provided. Prediction of sequestered soil carbon may be facilitated by using Raman spectral measurements, generating synthetic spectral measurements by physical simulation for training data, using spectral measurements at varying degrees of resolution (e.g. using satellite measurements bolstered by measurements from aerial and/or proximal sensors), and/or modeling uncertainty in the predictions. Synthetic and ground-truth modes of training may differ in the number and type of input provided.

### CROSS-REFERENCE TO RELATED APPLICATIONS

This application is a continuation of Patent Cooperation Treaty (PCT) application No. PCT/CA2022/051760 having an international filing date of 1 Dec. 2022, which in turn claims priority from, and for the purposes of the United States the benefit of 35 USC § 119 in respect of, U.S. provisional patent application No. 63/285,942 filed 3 Dec. 2021 and entitled Systems and Methods for Predicting Soil Carbon Content. All of the applications in this paragraph are hereby incorporated herein by reference.

### TECHNICAL FIELD

The present disclosure relates generally to a method of predicting soil carbon content in soils, and particularly to a method of predicting soil carbon content based on machine learning techniques.

### BACKGROUND

Quantifying soil carbon content in soils, such as agricultural soils, is a challenging technical problem with significance to global efforts against climate change. Soils can store carbon in various forms and such storage can be promoted by human interventions, such as certain agricultural or other land use-specific practices. Such practices are sometimes incentivized through the use of carbon credit markets, which benefit from the ability to quantify soil carbon content with high accuracy, high scale (e.g. across areas of tens to millions of square kilometers), and at low cost.

Traditional approaches for soil carbon quantification typically involves sending a sampling team to the area of interest, manually collecting soil samples, preparing the samples (e.g. via drying, sieving, and freezing), and shipping the prepared samples to a laboratory for characterization, e.g. via chemical analysis such as by dry combustion or acid treatment. Such approaches tend to be highly accurate, but tend to be laborious, costly, and slow—for instance, quantifying soil carbon content of one field can cost thousands of dollars and take months for results to come back, rendering it impractical for use at high scale.

Others have applied spectral approaches by measuring reflectance spectra of surface-level soils in the visible, near-IR, and/or short-wave IR spectral ranges and interpreting the measured spectra via a model to estimate soil carbon content. For example, Soriano-Disla et al. disclose soil spectroscopy methods for predicting soil carbon content involving obtaining diffuse reflectance infrared spectroscopy measurements of soils using handheld sensors in the visible-near infrared and mid-infrared spectral ranges and generating from those measurements predictions of soil carbon content (see Soriano-Disla et al., The Performance of Visible, Near-, and Mid-Infrared Reflectance Spectroscopy for Prediction of Soil Physical, Chemical, and Biological Properties. Appl. Spectrosc. Rev. 2014, 49, 139-186). Such methods can achieve reasonable accuracy and moderate scalability, but tend to involve calibrating based on large calibration datasets of laboratory-tested samples from each region in which spectral measurements are to be applied. Moreover, such methods can have reduced accuracy for certain forms of carbon, such as for soil organic matter.

Others have applied spectral approaches in remote sensing contexts, such as predicting soil carbon content based on satellite imagery. Such methods tend to be noisy and relatively lower-accuracy than the above-described techniques. For instance, Meng et al. disclose a remote sensing approach for predicting soil organic matter content at a regional scale using satellite reflectance hyperspectral data (see Meng et al., Soil Organic Matter Prediction Model with Satellite Hyperspectral Image Based on Optimized Denoising Method, Remote Sens. 2021, 13, 2273. https://doi.org/10.3390/rs13122273).

There is a general desire for soil carbon prediction systems and methods for generating predictions of soil carbon suitable to assessing sequestration of soil carbon with improved accuracy, at improved scale, and/or at reduced cost.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides systems and methods for predicting soil carbon content. The systems comprise one or more processors and a memory storing instructions which cause the one or more processors to perform operations comprising the methods. The methods are performed by a processor and comprise: obtaining an input spectral measurement of soil; generating a first prediction of sequestered soil carbon content for the soil by on a machine learning model (referred to herein as a “prediction model”) configured to generate predictions of sequestered soil carbon content based on input spectral measurement and having parameters trained over: synthetic sequestered soil carbon content training data and associated synthetic training spectral measurements; ground truth sequestered soil carbon content training data and associated ground truth training spectral measurements.

In some embodiments, the input spectral measurement comprises a Raman spectral measurement. In some embodiments, the Raman spectral measurement comprises a first spectral signature corresponding to mineral-associated organic material and a second spectral signature corresponding to particulate organic material, the method further comprising distinguishing at least a portion of the first spectral signature from the second spectral signature.

In some embodiments, the first prediction of sequestered soil carbon comprises a predicted measure of mineral associated organic matter content in the soil.

In some embodiments, the synthetic sequestered soil carbon content data comprises a measure of mineral associated organic matter content in a plurality of simulated soil samples, and the ground truth sequestered soil carbon content data comprises a measure of mineral associated organic matter content in a plurality of ground-truth soil samples.

In some embodiments the method further comprises generating a second prediction of non-sequestered soil carbon content for the soil.

In some embodiments the method further comprises generating a third prediction of total soil carbon content for the soil, the third prediction of total soil carbon content being based on a sum of soil carbon content of the first and second predictions.

In some embodiments, the input spectral measurement comprises a high-spatial-resolution spectral measurement and a low-spatial-resolution spectral measurement.

In some embodiments, the low-spatial-resolution spectral measurement comprises at least one of: satellite images and aerial images of an area of interest comprising a location of the soil.

In some embodiments, the high-resolution spectral measurement comprises an at-depth spectral measurement of the soil.

In some embodiments, the at-depth spectral measurement comprises a spectral measurement of a soil sample obtained at a depth of at least one of: about 0.01 m to 1 m, 0.1 m to 1 m, 0.25 m to 1 m, and 0.1 m to 10 m.

In some embodiments, the at-depth spectral measurement comprises a Raman spectral measurement.

In some embodiments, the synthetic training spectral measurements comprises a simulated spectral measurement generated by a physical soil simulation.

In some embodiments, at least one simulated spectral measurement for a physical soil simulation comprising first and second soil components is based on a sum of a first simulated spectral measurement for the first soil component and a second simulated spectral measurement for the second soil component.

In some embodiments, the synthetic training spectral measurements comprises a predicted spectral measurement generated by a generative machine learning model trained over soil properties and configured to predict spectral measurements based on soil properties.

In some embodiments, the simulated spectral measurement is based on a simulation of at least one of: a vibrational frequency and a vibrational intensity of molecules represented in the physical soil simulation.

In some embodiments, the first prediction comprises a measure of uncertainty.

In some embodiments, the machine learning model comprises a Bayesian ensemble neural network configured to provide the measure of uncertainty for each prediction.

In some embodiments, the ground truth sequestered soil carbon content training data corresponds to a plurality of ground truth training locations in an area of interest; the prediction model is configured to generate predictions of sequestered soil carbon content based on the input spectral measurement and a location identifier associated with the input spectral measurement, the prediction model having parameters trained over a plurality of ground-truth location identifiers associated with the ground truth training spectral measurements; and generating the first prediction of sequestered soil carbon content for the soil comprises generating the first prediction of sequestered soil carbon content for the location based on a location identifier for the soil associated with the input spectral measurement.

In some embodiments, generating the first prediction of sequestered soil carbon content comprises generating an intermediate representation by a first portion of the prediction model, combining the intermediate representation with one or more additional inputs to form a combined input, and generating the first prediction by a second portion of the prediction model based on the combined input.

In some embodiments, the first portion of the prediction model has parameters trained over synthetic sequestered soil carbon content training data and associated synthetic training spectral measurements; and the second portion of the prediction model has parameters trained over synthetic sequestered soil carbon content training data and associated synthetic training spectral measurements and ground-truth sequestered soil carbon content training data and associated ground-truth training spectral measurements.

Aspects of the present disclosure provide systems and methods for training a machine learning model to predict soil carbon content. The systems comprise one or more processors and a memory storing instructions which cause the one or more processors to perform operations comprising the methods. The method is performed by a processor and comprises: obtaining synthetic training spectral measurements based on simulated soil samples; generating a first plurality of predictions of sequestered soil carbon content by a machine learning model (referred to herein as a “prediction model”) based on the synthetic training spectral measurements; determining a first value of an objective function based on the first plurality of predictions of sequestered soil carbon content and synthetic sequestered soil carbon content training data associated with the synthetic training spectral measurements; and modifying parameters of the prediction model based on the first value of the objective function.

In some embodiments, the method comprises obtaining ground truth training spectral measurements based on soil samples from a plurality of sampling locations in an area of interest; generating a second plurality of predictions of sequestered soil carbon content for a plurality of training locations by the prediction model based on the ground truth training spectral measurements; determining a second value of an objective function based on the second plurality of predictions of sequestered soil carbon content and ground truth sequestered soil carbon content training data associated with the ground truth training spectral measurements; and modifying parameters of the prediction model based on the second value of the objective function.

In some embodiments, the ground truth sequestered soil carbon content data comprises a measure of mineral associated organic matter content in a plurality of soil samples at the plurality of training locations.

In some embodiments, the ground truth training spectral measurements comprise Raman spectral measurements.

In some embodiments, at least one ground truth training spectral measurement comprises a high-spatial-resolution spectral measurement and a low-spatial-resolution spectral measurement.

In some embodiments, the low-spatial-resolution spectral measurement comprises at least one of: a satellite image and an aerial image of at least a portion of the area of interest.

In some embodiments, the high-resolution spectral measurement comprises an at-depth spectral measurement of soil at one of the sampling locations.

In some embodiments, the at-depth spectral measurement comprises a spectral measurement of a soil sample obtained at a depth of at least one of: about 0.01 m to 1 m, 0.1 m to 1 m, 0.25 m to 1 m, and 0.1 m to 10 m.

In some embodiments, the at-depth spectral measurement comprises a Raman spectral measurement.

In some embodiments, the synthetic training spectral measurements comprise Raman spectral measurements.

In some embodiments, the method comprises generating the Raman spectral measurements by simulating at least one of: a vibrational frequency and a vibrational intensity of molecules represented in the simulated soil sample.

In some embodiments, the Raman spectral measurement comprises a first spectral signature corresponding to mineral-associated organic material and a second spectral signature corresponding to particulate organic material, the method further comprising distinguishing at least a portion of the first spectral signature from the second spectral signature.

In some embodiments, generating the first plurality of predictions of sequestered soil carbon content by the prediction model based on the synthetic training spectral measurements comprises, for at least one of the first plurality of predictions: generating a first intermediate representation by a first component of the prediction model based on at least one of the synthetic training spectral measurements; and generating the at least one of the first plurality of predictions based on the intermediate representation; and generating the second plurality of predictions of sequestered soil carbon content by the prediction model based on the ground-truth training spectral measurements comprises, for at least one of the second plurality of predictions: generating a second intermediate representation by the first component of the machine learning model based on at least one of the ground-truth training spectral measurements; combining the intermediate representation with one or more additional inputs to form a combined input; and generating the at least one of the second plurality of predictions by a second component of the prediction model based on the combined input.

In some embodiments, the one or more additional inputs comprises at least one of: a location identifier, a terrain gradient, an aspect, a convergence index, a hill shade, a multi-resolution right top flatness, a multi-resolution valley bottom flatness, a standardized water level index, a valley depth, an elevation, a different from the mean elevation, a deviation from the mean elevation, a relative terrain index, a profile curvature, and a planform curvature associated with the at least one of the ground-truth training spectral measurements.

In some embodiments, combining the intermediate representation with one or more additional inputs to form the combined input comprises concatenating the intermediate representation with at least one of the one or more additional inputs.

In some embodiments, at least one of the first plurality of predictions of sequestered soil carbon comprises a predicted measure of mineral associated organic matter content in the simulated soil samples.

In some embodiments, the synthetic sequestered soil carbon content data comprises a measure of mineral associated organic matter content in the simulated soil samples.

In some embodiments, the method comprises generating a prediction of non-sequestered soil carbon content for a soil sample.

In some embodiments, the method comprises generating a prediction of total soil carbon content for the soil sample based on a sum of soil carbon content of the first and second predictions.

In some embodiments, the synthetic training spectral measurements comprises a simulated spectral measurement generated by a physical soil simulation.

In some embodiments, obtaining synthetic training spectral measurements based on simulated soil samples comprises obtaining one or more soil properties and generating the physical soil simulation comprising the simulated spectral measurement based on the one or more soil properties.

In some embodiments, at least one simulated spectral measurement for the physical soil simulation of a simulation soil sample comprising first and second soil components is based on a sum of a first simulated spectral measurement for the first soil component and a second simulated spectral measurement for the second soil component.

In some embodiments, the synthetic sequestered soil carbon content training data comprises a predicted spectral measurement generated by a generative machine learning model trained over soil properties and configured to predict spectral measurements based on soil properties.

In some embodiments, obtaining synthetic training spectral measurements based on simulated soil samples comprises obtaining one or more soil properties and generating the predicted spectral measurement by the generative machine learning model based on the one or more soil properties.

In some embodiments, the generative machine learning model comprises a generative adversarial network.

In some embodiments, at least one of the first plurality of predictions comprises a measure of uncertainty.

In some embodiments, the machine learning model comprises a Bayesian ensemble neural network configured to provide the measure of uncertainty for each prediction.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1A schematically shows an example system for training an example machine learning model for predicting soil carbon content in a first training mode based on simulated soil samples according to the present disclosure.

FIG. 1B schematically shows an example system for training the example machine learning model of FIG. 1A in a second training mode based on ground-truth soil samples.

FIG. 2 schematically shows an example system for generating predictions of soil carbon content with the example machine learning model of FIG. 1A.

FIG. 3 is a flowchart of an example method for training an example machine learning model for predicting soil carbon content, such as by the systems of FIG. 1A and FIG. 1B.

FIG. 4 is a flowchart of an example method for generating predictions of soil carbon content with an example machine learning model for predicting soil carbon content, such as by the system of FIG. 2.

FIG. 5 shows a first exemplary operating environment that includes at least one computing system for performing methods described herein, such as the methods of FIGS. 3 and 4.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Soil carbon can be stored in soils relatively transiently, e.g. as particulate organic matter (POM), or relatively more durably, e.g. as mineral-associated organic matter (MAOM) or inorganic carbon. More durably stored forms of soil carbon tend to be preferred in contexts focused on addressing climate change. Such forms of soil carbon are referred to herein as “sequestered” soil carbon.

Systems and methods disclosed herein provide machine learning models which generate predictions of sequestered soil carbon content and are trained on synthetic training data with associated synthetic spectral measurements and (optionally, at least in some embodiments) ground truth training data and with associated ground truth spectral measurements. Systems and methods for training such machine learning models are also provided.

In some embodiments, prediction of sequestered soil carbon is facilitated by using Raman spectral measurements, which tend to be less sensitive to variations in angle of measurement, less sensitive to variations in soil water content, and more sensitive to differences in carbon fractions (e.g. MAOM vs. POM carbon) than at least some reflectance spectral measurements and in suitable circumstances may assist in improving accuracy of predictions of sequestered soil carbon. In some embodiments, prediction of sequestered soil carbon is facilitated by generating synthetic spectral measurements by physical simulation for training data, which in suitable circumstances may assist with reducing the quantity of ground-truth samples required for training and/or with improving accuracy of predictions by trained models. In some embodiments, prediction of sequestered soil carbon is facilitated by using spectral measurements at varying degrees of resolution (e.g. using satellite measurements bolstered by measurements from aerial and/or proximal sensors), and/or modeling uncertainty in the predictions.

### Systems for Training a Sequestered Soil Carbon Content Prediction Model

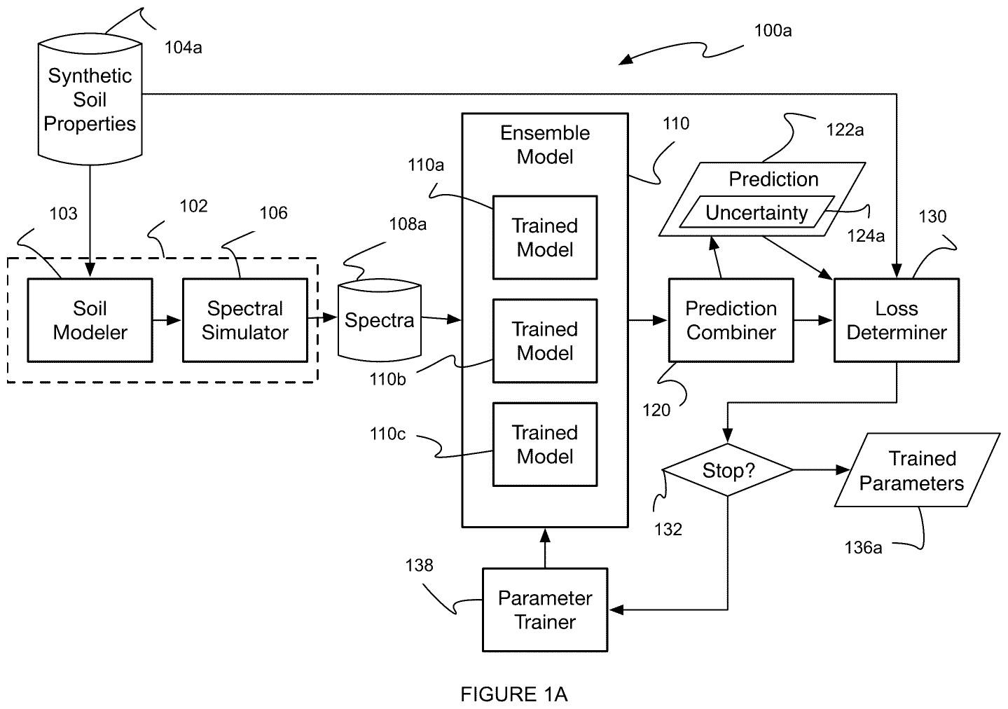

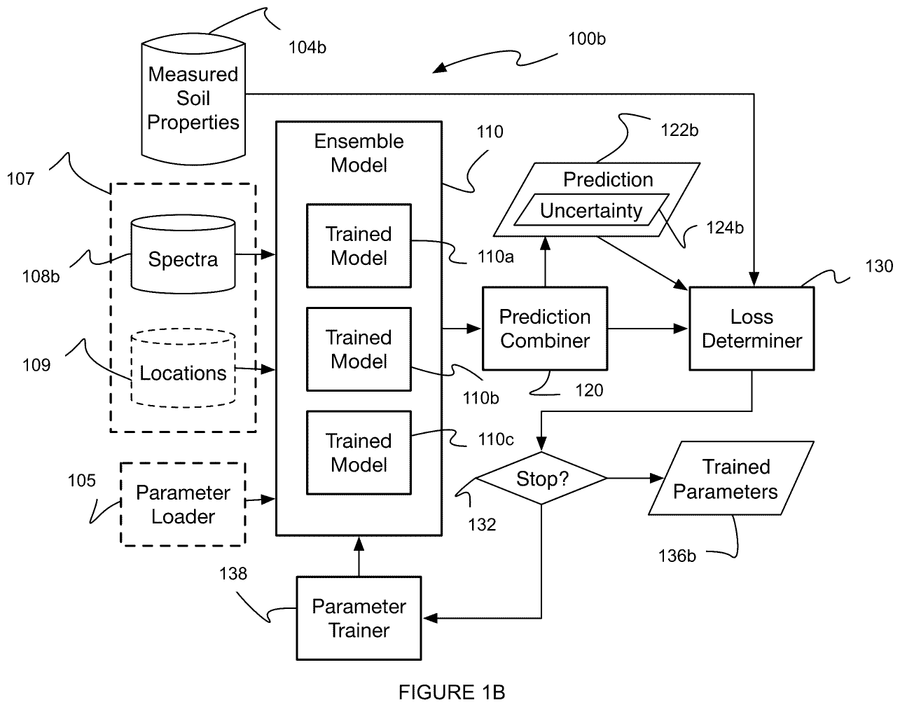

FIG. 1A schematically shows an example system 100a for training an example machine learning model 110 for predicting soil carbon content in a first training model based on simulated soil samples according to the present disclosure.

In some embodiments, system 100a comprises a synthetic soil spectral generator 102 for generating synthetic spectra of simulated soil samples. Synthetic soil spectral generator 102 receives synthetic soil properties 104a and generates synthetic soil spectra 108a based on synthetic soil properties 104a. Synthetic soil properties 104a may comprise a representation of carbon content, e.g. specifying 5% carbon content for a simulated soil sample, specifying 3% carbon content stored as mineral-associated organic matter and 2% carbon content stored as particulate organic matter, and/or as other carbon fractions of a simulated soil sample. Synthetic soil properties 104a may additionally, or alternatively, comprise representations of other soil properties, such as fractions of nitrogen, potassium, sulfur, hydrogen, nitrate, ammonia, phosphate, aluminum, iron, phosphorus, calcium, magnesium, and/or sodium content; phospholipid fatty acid content; pH; carbon-nitrogen ratio; Haney test inputs and outputs (which may include certain of the foregoing and/or other features such as water-soluble organic carbon and/or organic nitrogen); and/or other soil properties.

Synthetic soil spectral generator 102 may comprise a soil modeler 103 and a spectral simulator 106 (e.g. as shown in the example embodiment of FIG. 1A). Soil modeler 103 receives synthetic soil properties 104a for a simulated soil sample, such as a representation of carbon content and/or other soil properties as described elsewhere herein, and generates a soil model for the simulated soil sample based on the synthetic soil properties 104a. The soil model may comprise, for example, a representation of atoms, functional groups, molecular building blocks, molecules, and/or groups of molecules in the simulated soil sample. For instance, soil modeler 103 may comprise molecular building blocks as provided by Vienna Soil Organic Modeler 2 (e.g. as described by Escalona et al., Vienna Soil Organic Matter Modeler 2 (VSOMM2), J. Mol. Graph. Model. (2021) 103, 107817, doi: 10.1016/j.jmgm.2020.107817).

Spectral simulator 106 generates one or more simulated spectral measurements 108a for the simulated soil sample based on the soil model. In some embodiments, the simulated spectral measurement is generated by physical simulation of soil, e.g. by simulating molecular dynamical properties and/or quantum mechanical properties of the simulated soil sample, normal mode analysis (e.g. such as complex-based normal mode analysis). In some embodiments, spectral simulator 106 simulates Raman spectra for the simulated soil sample by simulating vibrational frequency and vibrational intensity of molecules represented in the soil model based on polarizability of the molecules. In some embodiments, spectral simulator 106 simulates IR spectra for the simulated soil sample by simulating vibrational frequency and vibrational intensity of molecules represented in the soil model based on changes in dipole moments of the molecules. For instance, molecular dynamics properties and/or quantum mechanical properties may be simulated via the GROMACS and/or Gaussian™ (from Gaussian, Inc.) computational chemistry software packages. In some embodiments, physical soil simulation comprises simulating interactions between molecules based on normal mode analysis, such as complex-based normal mode analysis, based on elastic network models. For instance, molecular interactions may be modeled based on an anisotropic network model. In some embodiments, spectral simulator 106 simulates more than one type of spectra; for example, spectral simulator 106 may simulate two or more of: Rayleigh, Raman, IR, reflectance, and/or other types of spectra.

In some embodiments, synthetic soil spectral generator 102 generates models of soil components for a simulated soil sample, generates synthetic spectra for at least some of the soil components, and synthesizes a synthetic spectral measurement by combining the synthetic spectra for the soil components. For example, soil modeler 103 may receive a set of soil properties 104a (e.g. indicating 5% soil carbon content) and generate a soil model comprising several soil components (e.g. molecules). Spectral simulator 106 may generate (and/or receive from a cache, e.g. if it has previously generated) synthetic spectral measurements for some or all of the soil components, and may combine those spectral measurements (e.g. by summation) to form a synthetic spectral measurement 108a for the simulated soil sample. Although some interactions between molecules may be lost in such a modeling approach, synthetic spectral measurements 108a may be more efficiently and quickly produced. At least in the contexts of Raman and IR spectra (though not limited to those contexts), loss in accuracy of the synthetic soil spectral measurements 108a under such an approach may be relatively small, and perhaps even negligible in suitable circumstances. Synthetic soil spectral measurements 108a may comprise one or more of Raman, IR, reflectance, and/or another type of spectral measurement.

In some embodiments, synthetic soil spectral generator 102 comprises a machine learning model. The machine learning model may comprise a generative machine learning model, such as a generative adversarial network (GAN), an invertible network, and/or any other suitable model. The machine learning model may be trained over ground-truth and/or simulated soil sample data to generate synthetic spectral measurements 108a based on one or more synthetic soil properties 104a. In at least some embodiments, soil properties 104a comprise a measure of sequestered soil carbon in a form corresponding to a measure of sequestered soil carbon provided by prediction 122a to facilitate training of machine learning model 110. Training machine learning model 122a may be performed separately from training the machine learning model of soil spectral generator 102 and may comprise comparing predicted sequestered soil carbon content of prediction 122a for a given simulated soil sample to the sequestered soil carbon content of soil properties 104a for that simulated soil sample and determining a loss (e.g. as described elsewhere herein with reference to loss determiner 130) based on that comparison.

Machine learning model 110 receives a synthetic spectral measurement 108a and generates a prediction 122a of soil carbon content for the simulated soil sample associated with the received synthetic spectral measurement 108a. Prediction 122a may additionally, or alternatively, comprise predictions of other soil properties, such as nitrogen, potassium, sulfur, hydrogen, nitrate, ammonia, phosphate, aluminum, iron, phosphorus, calcium, magnesium, and/or sodium content; phospholipid fatty acid content; pH; carbon-nitrogen ratio; Haney test inputs and outputs (which may include certain of the foregoing and/or other features such as water-soluble organic carbon and/or organic nitrogen); and/or other soil properties.

In some embodiments, including the depicted embodiment, the machine learning model comprises an ensemble model comprising a plurality of sub-models 110a, 110b, 110c, etc. (An ensemble machine learning model 110 may comprise any number of sub-models.) In some embodiments, predictions of sub-models 110a, 110b, 110c are combined by prediction combiner 120 to generate prediction 122a, e.g. by determining an average (e.g. a weighted average) and/or by any other suitable combination technique in the art.

In at least some embodiments, machine learning model 110 is configured to provide a measure of uncertainty 124a for prediction 122a. For instance, machine learning model 110 may comprise a Bayesian ensemble neural network configured to provide a measure of uncertainty 124a for prediction 122a based on predictions by sub-models 110a, 110b, 110c (e.g. based on a variance of such predictions by sub-models 110a, 110b, 110c).

In at least some embodiments, loss determiner 130 determines a loss value of an objective function for a plurality of predictions 122a. For a given plurality of predictions 122a based on a corresponding plurality of synthetic spectral measurements 108a (from which machine learning model 110 generated predictions 122a), the loss value for the objective function may be based on predictions 122a and the synthetic soil properties 104a from which the corresponding plurality of synthetic spectral measurements 108a were generated. For instance, synthetic soil properties 104a, and/or information derived therefrom (e.g. soil carbon content of soil models generated therefrom by soil modeler 103), may be used as labels for the purpose of determining a value of an objective function by loss determiner 130 in the course of training machine learning model 110.

Parameter trainer 138 trains parameters of model 110 based on the value of the objective function determined by loss determiner 130. Training parameters of model 110 may comprise determining updated parameters via an optimization algorithm, such as gradient descent. For instance, parameter trainer 138 may determine updated parameters based on a gradient of the objective function with respect to the parameters (sometimes denoted ∇μ/∇θ for an objective function u and parameters θ).

System 100a may train parameters of model 110 on synthetic spectral measurements 108a over a plurality of batches and/or epochs and may stop by applying a stopping criterion 132. Stopping criterion 132 may comprise completing a predetermined (e.g. user-provided) number of batches and/or epochs, achieving a target loss value (at loss determiner 130), achieving a target gradient (at parameter trainer 138), and/or any other suitable criterion. As one non-limiting example, in the example embodiment of FIG. 1A, stopping criterion 132 is based on a loss value determined by loss determiner 130.

When system 100a stops training model 110, the parameters of model 110 as trained by parameter trainer 138 may be output, stored, or otherwise provided as trained parameters 136a.

In some embodiments, system 100a trains only a portion of model 110. For example, system 100a may train a first portion of model 110 which receives spectral measurements as input but which does not receive certain other inputs of model 110. Model 110 may comprise a second portion which receives input based on the output of the first portion and one or more further inputs, such as location identifiers (e.g. location identifiers 109, described in greater detail with reference to FIG. 1B). For example, the first component of model 110 may comprise a convolutional neural network where convolutions (e.g. one-dimensional convolutions) are applied to input spectral measurements, such as synthetic spectral measurements 108a and ground-truth spectral measurements 108b (described in greater detail with reference to FIG. 1B). The output of the first component may be transformed to a prediction 122a by applying a prediction transformation, such as by transforming the output of the first component by a fully-connected layer. The first component network and prediction transformation may be trained by system 100a as described above.

Where there is a desire to train model 110 over further inputs which are not provided for synthetic spectral measurements 108b and/or which are not provided by system 100a, the output of the first component may be combined with such further inputs (e.g. location identifiers, terrain representations, and/or other covariate data) and passed through the second component of model 110 to generate prediction 122b, as described in greater detail with reference to FIG. 1B. Combination of inputs may comprise concatenation, a non-linear combination (e.g. via a neural network), and/or any other suitable combination. The second component may, for example, comprise a second neural network (e.g. comprising a fully connected layer) trained to generate prediction 122b as output. The prediction transformation for generating prediction 122a is not necessarily applied when training the second component so as to preserve representational richness.

In some embodiments, system 100a trains all of model 110. For such embodiments where model 110 receives input other than spectral measurements, synthetic spectral measurements 108a may be associated with further synthetic inputs, such as synthetic location identifiers and/or any other inputs expected by model 110. Further synthetic inputs may be predetermined, e.g. provided by a user, and/or imputed by system 100a. For example, system 100a may determine a synthetic location for a given spectral measurement 108a by generating a location based on an input spectral measurement via a machine learning model trained over ground-truth spectral measurements and associated location identifiers (such as ground-truth spectral measurements 108b and location identifiers 109, described in greater detail with reference to FIG. 1B, and/or based on other spectral measurements and location identifiers, such as those obtained from regional soil characteristics datasets). In some embodiments, a synthetic location for a given spectral measurement 108a is based on synthetic soil properties 104a for the given spectral measurement 108a. For example, system 100a may associate synthetic soil properties 104a with a location and/or a region based on similarity of synthetic soil properties 104a to soil properties associated with the location and/or region, e.g. by clustering locations and/or regions in a soil properties database based on soil properties, associating synthetic soil properties 104a for the given spectral measurement 108a with a cluster of locations and/or regions, and selecting a location from the cluster of locations and/or regions (e.g. by random sampling and/or any other suitable selection approach) to use as the synthetic location. A synthetic location identifier for the synthetic location (e.g. longitude and latitude coordinates) may be provided to model 110 as input.

In some embodiments, intermediate features are generated based on synthetic soil measurements 108a and/or other inputs to machine learning model 110 and may be provided as input to machine learning model 110. For example, intermediate features may be generated by applying spectral transformations, dimensionality transformations, and/or other transformations to synthetic soil measurements 108a. Such intermediate features may be provided to machine learning model 110 instead of, or in addition to, synthetic soil measurement 108a.

FIG. 1B schematically shows an example system 100b for training machine learning model 110 in a second training mode based on ground-truth soil samples. Systems 100a and 100b may be provided by a system 100 and/or by different systems. In some embodiments, model 110 is initialized with trained parameters 136a by parameter loader 105 and system 100b further trains parameters of model 110 to generate trained parameters 136b (e.g. model 110 may be “pre-trained” by system 110 and “fine-tuned” by model 100b). In some embodiments, model 110 is trained by systems 100a and 100b in parallel and/or by interleaving, e.g. by alternating between training by system 100a and 100b.

System 100b trains machine learning model 110 over ground-truth spectral measurements 108b of the ground-truth soil samples. Ground truth spectral measurements 108b may comprise, for example, one or more of: Rayleigh, Raman, IR, and/or other spectral measurements. In some embodiments, ground truth spectral measurements 108b comprise Raman spectral measurements. Ground-truth spectral measurements 108b are associated with ground-truth training data comprising measured soil properties 104b of the ground-truth soil samples. Measured soil properties 104b may comprise, for example, measures of sequestered soil carbon content obtained via chemical analysis and/or any other suitable approach for characterizing sequestered soil carbon content. Measures of sequestered soil carbon content may comprise measures of soil carbon based on whether the soil carbon is mineral-associated, permanent, stable, or otherwise durable. For instance, measured soil properties 104b may comprise a measure of mineral associated organic matter (MAOM) content in ground-truth soil samples. Measured soil properties 104b may additionally, or alternatively, comprise measures of other soil properties, such as non-sequestered soil carbon content (e.g. particulate organic matter, or POM, content); total soil carbon content; nitrogen, potassium, sulfur, hydrogen, nitrate, ammonia, phosphate, aluminum, iron, phosphorus, calcium, magnesium, and/or sodium content; phospholipid fatty acid content; pH; carbon-nitrogen ratio; Haney test inputs and outputs (which may include certain of the foregoing and/or other features such as water-soluble organic carbon and/or organic nitrogen); and/or other soil properties.

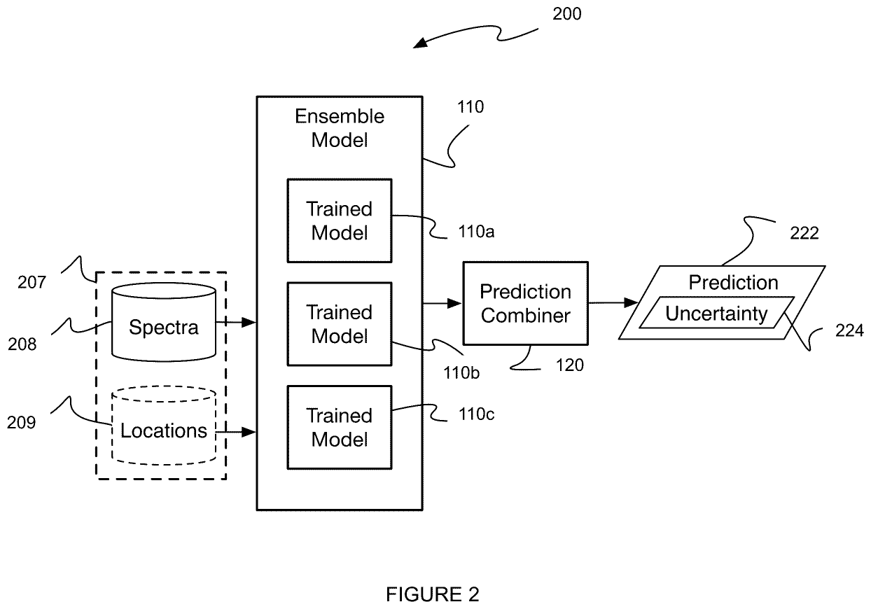

Spectral measurements 108b may be associated with other inputs for model 110, together forming inputs 107. In some embodiments, inputs 107 comprise location identifiers 109 associated with ground-truth spectral measurements 108b. Location identifiers 109 represent locations of ground-truth soil samples, such as a latitude and longitude of the location where a ground-truth soil sample was collected. In at least some embodiments, inputs 107 have the same form as inputs 207 (described elsewhere herein with respect to FIG. 2).

System 100b generates predictions 122b via machine learning model 110 based on inputs 107, including ground-truth spectral measurements 108b. Machine learning model 110 receives a ground-truth spectral measurement 108b and other inputs 107 (if any) and generates a prediction 122b of soil carbon content in the simulated soil sample associated with the received ground-truth spectral measurement 108b and other inputs 107 (if any). Prediction 122b may additionally, or alternatively, comprise predictions of other soil properties, such as nitrogen, potassium, sulfur, hydrogen, nitrate, ammonia, phosphate, aluminum, iron, phosphorus, calcium, magnesium, and/or sodium content; phospholipid fatty acid content; pH; carbon-nitrogen ratio; Haney test inputs and outputs (which may include certain of the foregoing and/or other features such as water-soluble organic carbon and/or organic nitrogen); and/or other soil properties.

In at least some embodiments, machine learning model 110 is configured to provide a measure of uncertainty 124b for prediction 122b, e.g. substantially as described elsewhere herein. For instance, machine learning model 110 may comprise a Bayesian ensemble neural network configured to provide a measure of uncertainty 124b for prediction 122b based on predictions by sub-models 110a, 110b, 110c (e.g. based on a variance of such predictions by sub-models 110a, 110b, 110c).

System 100b trains parameters of model 110 based on prediction 122b. In at least some embodiments, system 100b trains parameters of model 110 based on prediction 122b substantially as described above with reference to system 100a and prediction 122a of FIG. 1A. System 100b provides trained parameters 136b for model 110 as a result of said training.

In some embodiments, spectral measurements 108b comprise high-spatial-resolution spectral measurements and low-spatial-resolution spectral measurements. For example, low-spatial-resolution spectral measurements may comprise satellite images and/or aerial images of an area of interest. Such images may be associated with location identifiers on the basis of which system 100b may determine location identifiers for spectral measurements 108b (e.g. further based on a location of a spectral measurement within the image and a resolution of the image).

As another example, high-resolution spectral measurements may comprise at-depth spectral measurement of soil, such as spectral measurements of soil samples obtained at a depth of about 0.01 m to 1 m, 0.1 m to 1 m, 0.2 m to 1 m, 0.3 m to 1 m, 0.4 m to 1 m, 0.5 m to 1 m, 0.25 m to 1 m, 0.25 m to 0.75 m, 0.25 m to 0.5 m, 0.5 m to 1 m, 0.1 m to 10 m, 0.1 m to 5 m, and/or 0.1 m to 2 m.

Spectral measurements may comprise Rayleigh, Raman, IR, and/or other spectral measurements. For example, in some embodiments the high-resolution spectral measurements comprise Raman spectral and/or IR spectral measurements of soil samples obtained proximally at depth (and may optionally include Rayleigh spectral measurements), and the low-resolution spectral measurements comprise Rayleigh measurements obtained aerially. For instance, a proximal sensor (e.g. a handheld sensor and/or a vehicle-mounted sensor) may measure spectra of soil at various locations throughout the area of interest. As another example, soil samples may be collected from the area of interest, returned to a lab, and their spectra may be measured there with laboratory spectrometers. (Other soil properties may optionally be measured as well.)

As noted above, in some embodiments, such as embodiments where inputs 107 provide more information than is provided during training by system 100a, model 110 may comprise first and second components. The first component may be trained by system 100a and the second component may be trained by system 100b. Such training by system 100b may comprise generating an intermediate representation by the first component, as trained by system 100a, combining inputs 107 not received by the first component with the intermediate representation (e.g. by concatenation), and transforming such combination to prediction 122b by the second component of model 110. The second component may, for example, comprise a second neural network (e.g. comprising a fully connected layer).

In some embodiments, training model 110 may comprise performing semi-supervised training. Such training may comprise providing unlabeled data, e.g. by providing at least some soil measurements 108a and/or 108b without providing and/or training over ground-truth training data such as measured soil properties 104b. Such training may comprise performing techniques such as self-training, co-training, and/or autoencoder-based pretraining to model 110 based on soil measurements 108a and/or 108b.

Systems for Predicting Sequestered Soil Carbon Content with a Model

FIG. 2 schematically shows an example system 200 for generating predictions of soil carbon content with the example machine learning model 110 of FIGS. 1A and 1B.

System 200 obtains input data 207, e.g. from a user, from a data store, or via any suitable source. Input data 207 comprises an input spectral measurement 208 of a soil sample. Input data 207 may optionally comprise other data, such as a location identifier 209 associated with input spectral measurement 208, e.g. substantially as described elsewhere herein with reference to location identifier 109 and ground-truth spectral measurement 108b. Input data 207 may alternatively or additionally comprise representations of terrain characteristics, such as slopes/gradients, aspect, convergence index, hill shade, multi-resolution right top flatness, multi-resolution valley bottom flatness, standardized water level index, valley depth, elevation, different from the mean elevation, deviation from the mean elevation, relative terrain index, profile curvature, planform curvature, and/or other characteristics of terrain at the location of the soil sample.

System 200 generates, by machine learning model 110 and based on the trained parameters of machine learning model 110 (such as trained parameters 136a, 136b) a prediction 222 of sequestered soil carbon content for the soil sample associated with spectral measurements 208. The trained parameters of machine learning model 110 were trained over synthetic sequestered soil carbon content training data and associated synthetic training spectral measurements (e.g. substantially as described above with respect to synthetic spectral measurements 108a and system 100a of FIG. 1A) and ground truth sequestered soil carbon content training data and associated ground truth training spectral measurements (e.g. substantially as described above with respect to ground-truth spectral measurements 108b and system 100b of FIG. 1B). Model 110 may comprise an ensemble model and system 200 may comprise a prediction combiner 120 for combining predictions of sub-models 110a, 110b, 110c to form prediction 222, e.g. substantially as described elsewhere herein.

In at least some embodiments, machine learning model 110 is configured to provide a measure of uncertainty 224 for prediction 222, e.g. substantially as described elsewhere herein.

For instance, machine learning model 110 may comprise a Bayesian ensemble neural network configured to provide a measure of uncertainty 224 for prediction 222 based on predictions by sub-models 110a, 110b, 110c (e.g. based on a variance of such predictions by sub-models 110a, 110b, 110c).

### Methods for Training a Sequestered Soil Carbon Content Prediction Model

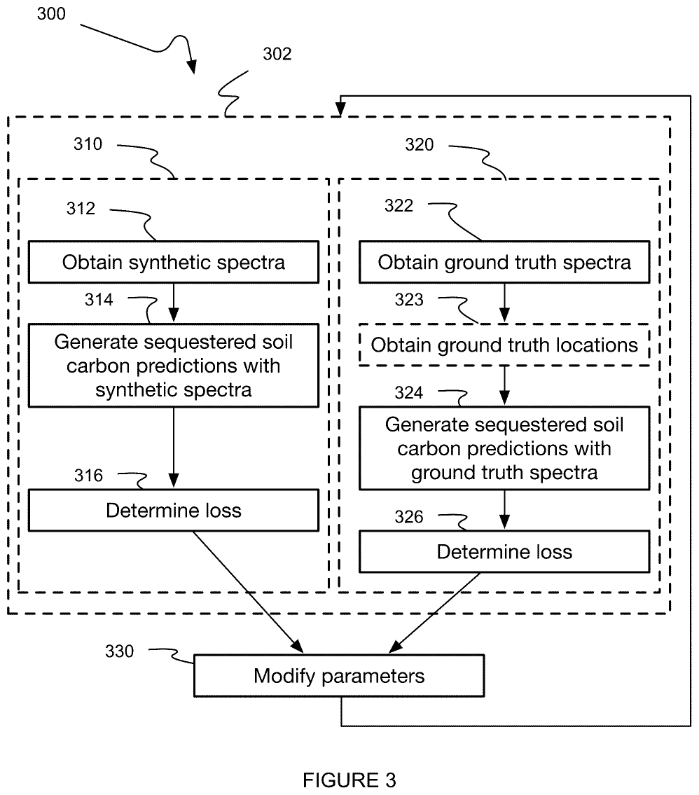

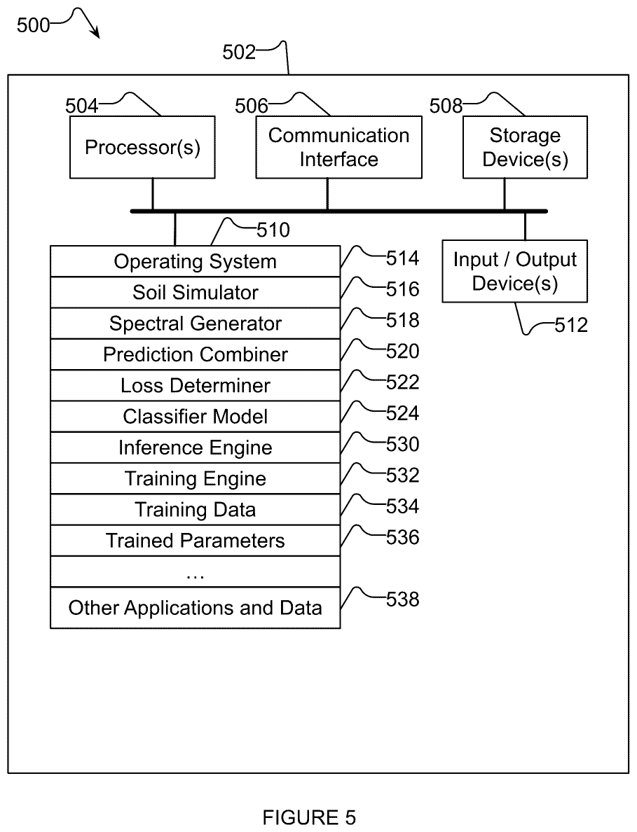

FIG. 3 is a flowchart of an example method 300 for training an example machine learning model (such as machine learning model 110) for predicting sequestered soil carbon content, such as by the systems 100a, 100b of FIGS. 1A and 1B. Method 300 is performed by a processor, e.g. as described with reference to system 500 of FIG. 5.

In some embodiments, method 300 comprises acts 302 for training the machine learning model over synthetic and ground-truth spectral measurements. Acts 302 may comprise acts 310 for training the machine learning model over synthetic spectral measurements and/or acts 320 for training the machine learning model over ground-truth spectral measurements. Acts 310 and 320, where both are provided by method 300, may be performed in series (e.g. by performing acts 310 to completion and then performing acts 320 to completion), in parallel (e.g. by training the machine learning model on both synthetic and ground-truth spectral measurements in a given batch), by interleaving (e.g. by training via acts 310, followed by acts 320, followed by acts 310, followed by acts 320, and so on), and/or in any other suitable order. Such training, in the case of each of acts 310 and 320, may comprise modifying parameters as described with reference to act 330.

Turning to acts 310, at act 312, the processor obtains synthetic spectral measurements for a plurality of simulated soil samples, e.g. as described with reference to synthetic spectral measurements 108a of FIG. 1A. The synthetic spectral measurements may comprise reflectance, IR, Raman, and/or other forms of spectra. In some embodiments, the synthetic spectral measurements comprise Raman spectral measurements.

At act 314, the processor generates a plurality of predictions of sequestered soil carbon content for the simulated soil samples obtained at act 312. The processor generates the plurality of predictions based on a machine learning model (referred to herein for disambiguation as a “prediction model”) based on the synthetic spectral measurements, and optionally other inputs (e.g. location identifiers, terrain representations, and/or other covariate data). Generating the plurality of predictions may comprise, for example, transforming the synthetic spectral measurements based on trained parameters of the prediction model to form a prediction of sequestered soil carbon content for the simulated soil sample, e.g. as described with reference to prediction 122a of FIG. 1A. In some embodiments act 314 comprises generating an intermediate prediction suitable for use by acts 324 and/or 404 and transforming the intermediate prediction to a prediction, e.g. as described elsewhere herein with reference to FIGS. 1A and 1B. The prediction model may comprise, for example, a regressor and/or classifier machine learning model.

At act 316, the processor determines a value of an objective function based on the plurality of predictions of sequestered soil carbon content and synthetic sequestered soil carbon content training data associated with the synthetic spectral measurements. For example, the synthetic sequestered soil carbon content training data may comprise a measure of sequestered soil carbon content (and/or other characteristics), e.g. as described above with reference to synthetic soil properties 104a. The processor may determine the value of the objective function as described elsewhere herein with reference to loss determiner 130 of FIG. 1A.

In some embodiments, act 302 of method 300 comprises acts 320 for training parameters of the machine learning model based on ground-truth spectral measurements. For example, at act 322 the processor obtains ground-truth spectral measurements for a plurality of ground-truth soil samples, e.g. as described with reference to ground-truth spectral measurements 108b of FIG. 1B.

Optionally, at act 323, the processor obtains location identifiers associated with the ground-truth spectral measurements obtained at act 322. For example, the processor may obtain location identifiers 109 associated with ground-truth spectral measurements 108b, e.g. as described with reference to FIG. 1B.

At act 324, the processor generates a plurality of predictions of sequestered soil carbon content for the soil samples based on the ground truth training spectral measurements obtained at act 322. The processor generates the plurality of predictions based on a prediction model based on the ground-truth spectral measurements, such as by transforming the ground-truth spectral measurements based on trained parameters of the prediction model to form a prediction of sequestered soil carbon content for the simulated soil sample, e.g. as described with reference to prediction 122b of FIG. 1B.

In some embodiments, act 324 comprises providing the prediction model with additional inputs, such as location identifiers associated with the ground-truth spectral measurements. Additional inputs may alternatively or additionally comprise representations of terrain characteristics, such as slopes/gradients, aspect, convergence index, hill shade, multi-resolution right top flatness, multi-resolution valley bottom flatness, standardized water level index, valley depth, elevation, different from the mean elevation, deviation from the mean elevation, relative terrain index, profile curvature, planform curvature, and/or other characteristics of terrain at the location of the soil sample. For example, act 324 may comprise generating an intermediate representation based on a portion of the input data (e.g. as described elsewhere herein with reference to act 314), combining the intermediate representation with additional inputs, and generating a prediction based on the combined inputs (e.g. as described elsewhere herein with reference to FIGS. 1A and 1B.

At act 326, the processor determines a value of an objective function based on the plurality of predictions of sequestered soil carbon content and ground-truth sequestered soil carbon content training data associated with the ground-truth spectral measurements. For example, the ground-truth sequestered soil carbon content training data may comprise a measure of sequestered soil carbon content (and/or other characteristics), e.g. as described above with reference to ground-truth soil properties 104b. The processor may determine the value of the objective function as described elsewhere herein with reference to loss determiner 130 of FIG. 1B.

At act 330, the processor modifies parameters of the prediction model based on one or more values of the objective functions obtained at acts 316 and/or 326. Modifying the parameters of the prediction model may comprise, for example, determining updated parameters via an optimization algorithm, such as gradient descent, e.g. as described with reference to parameter trainer 138 of FIGS. 1A and 1B.

Methods for Predicting Sequestered Soil Carbon Content with a Model

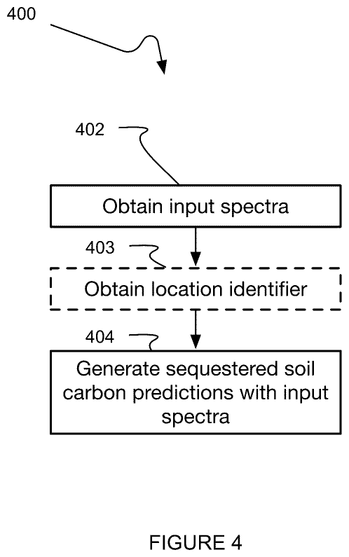

FIG. 4 is a flowchart of an example method 400 for generating predictions of soil carbon content with an example machine learning model (such as machine learning model 110) for predicting sequestered soil carbon content, such as by the system 200 of FIG. 2. Method 400 is performed by a processor, e.g. as described with reference to system 500 of FIG. 5.

At act 402, the processor obtains input spectral measurements for soil at a location, e.g. as described with reference to input spectral measurements 208 of FIG. 2.

Optionally, at act 403, the processor obtains a location identifier associated with the input spectral measurement obtained at act 402. For example, the processor may obtain a location identifier 209 associated with input spectral measurement 208, e.g. as described with reference to FIG. 2.

At act 404, the processor generates prediction of sequestered soil carbon content for the soil associated with the input spectral measurement obtained at act 402. The processor generates the prediction by a prediction model based on the input spectral measurement. The prediction model has trained parameters trained over synthetic sequestered soil carbon content training data (such as synthetic soil properties 104a) and associated synthetic training spectral measurements (such as synthetic spectral measurements 108a) and, optionally, based on ground-truth sequestered soil carbon content training data (such as ground-truth soil properties 104b) and associated ground-truth training spectral measurements (such as ground-truth spectral measurements 108b). For instance, the trained parameters of the prediction model may be trained as described according to method 300. Training the trained parameters of the prediction model is not necessarily included within the scope of method 400, although such acts may optionally be included.

Act 404 optionally comprises providing the prediction model with additional inputs, such as location identifiers, terrain representations, and/or other covariate data as described in greater detail elsewhere herein. Generating a prediction may comprise, for example, transforming the input spectral measurements based on trained parameters of the prediction model to form a prediction of sequestered soil carbon content for the soil associated with the input spectral measurement, e.g. as described with reference to prediction 222 of FIG. 2. In some embodiments act 404 comprises generating an intermediate prediction by a first component of the prediction model based on the input spectral measurement (and, optionally, other inputs) and generating a prediction based on the intermediate representation and further inputs by a second component of the prediction model, e.g. as described in greater detail elsewhere herein.

In at least some embodiments, act 404 comprises generating a measure of uncertainty for the prediction. For instance, the prediction model may comprise a Bayesian ensemble neural network configured to provide a measure of uncertainty for predictions based on predictions by sub-models of the ensemble model (such as sub-models 110a, 110b, 110c of model 110), e.g. as described in greater detail elsewhere herein.

### Computer System

FIG. 5 illustrates a first exemplary operating environment 500 that includes at least one computing system 502 for performing methods described herein. System 502 may be any suitable type of electronic device, such as, without limitation, a mobile device, a personal digital assistant, a mobile computing device, a smart phone, a cellular telephone, a handheld computer, a server, a server array or server farm, a web server, a network server, a blade server, an Internet server, a work station, a mini-computer, a mainframe computer, a supercomputer, a network appliance, a web appliance, a distributed computing system, multiprocessor systems, or combination thereof. System 502 may be configured in a network environment, a distributed environment, a multi-processor environment, and/or a stand-alone computing device having access to remote or local storage devices.

A computing system 502 may include one or more processors 504, a communication interface 506, one or more storage devices 508, one or more input and output devices 512, and a memory 510. A processor 504 may be any commercially available or customized processor and may include dual microprocessors and multi-processor architectures. A processor 504 may comprise a CPU, a GPU, and/or any other suitable processor. Terms such as “a processor” and “the processor” include a plurality of processors. The communication interface 506 facilitates wired or wireless communications between the computing system 502 and other devices. A storage device 508 may be a computer-readable medium that does not contain propagating signals, such as modulated data signals transmitted through a carrier wave. Examples of a storage device 508 include without limitation RAM, ROM, EEPROM, flash memory or other memory technology, CD-ROM, digital versatile disks (DVD), or other optical storage, magnetic cassettes, magnetic tape, magnetic disk storage. In at least some embodiments such embodiments of storage device 508 do not contain propagating signals, such as modulated data signals transmitted through a carrier wave. There may be multiple storage devices 508 in the computing system 502. The input/output devices 512 may include a keyboard, mouse, pen, voice input device, touch input device, display, speakers, printers, etc., and any combination thereof.

The memory 510 may be any non-transitory computer-readable storage media that may store executable procedures, applications, and data. The computer-readable storage media does not pertain to propagated signals, such as modulated data signals transmitted through a carrier wave. It may be any type of non-transitory memory device (e.g., random access memory, read-only memory, etc.), magnetic storage, volatile storage, non-volatile storage, optical storage, DVD, CD, floppy disk drive, etc. that does not pertain to propagated signals, such as modulated data signals transmitted through a carrier wave. The memory 510 may also include one or more external storage devices or remotely located storage devices that do not pertain to propagated signals, such as modulated data signals transmitted through a carrier wave.

The memory 510 may contain instructions, components, and data. A component is a software program that performs a specific function and is otherwise known as a module, program, engine, and/or application. The memory 510 may include an operating system 514, a soil simulator 516 (e.g. providing a soil simulator 102), a spectral generator 518 (e.g. providing a spectral generator 106), a prediction combiner 520 (e.g. providing a prediction combiner 120), a loss determiner 522 (e.g. providing a loss determiner 130), a classifier model 524 (e.g. providing an ensemble model 110), an inference engine 530 (e.g. for performing inference via method 400), a training engine 532 (e.g. for performing training via method 300), training data 534 (e.g. comprising synthetic and/or ground-truth spectral measurements, soil properties, and/or other data as described in greater detail elsewhere herein), trained parameters 536 (e.g. comprising parameters 136a and/or 136b), and other applications and data 538.

Depending on the embodiment, some such elements may be wholly or partially omitted. For example, an embodiment configured for inference (e.g. via method 400 and/or system 200) but not necessarily for training might omit training data 534, soil simulator 516, spectral generator 518, loss determiner 522, and/or training engine 532. As another example, an embodiment configured for training (e.g. via method 300 and/or systems 100a, 100b) but not necessarily for inference might omit inference engine 530. In some embodiments configured for training, memory 510 may omit trained parameters 536 prior to starting training and may generate such trained parameters 536 over the course of training. Some or all components may be obtained via an input device 512, communication interface 506, and/or storage device 508.

### CONCLUSION

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions and sub-combinations as are within their true spirit and scope.

What is claimed is:

1. A method for predicting soil carbon content, the method performed by a processor and comprising:
obtaining an input spectral measurement of soil;
generating a first prediction of sequestered soil carbon content for the soil by a machine learning model configured to generate predictions of sequestered soil carbon content based on input spectral measurement and having parameters trained over:
synthetic sequestered soil carbon content training data and associated synthetic training spectral measurements;
ground truth sequestered soil carbon content training data and associated ground truth training spectral measurements.

2. The method according to claim 1 wherein the input spectral measurement comprises a Raman spectral measurement.

3. The method according to claim 2 wherein the Raman spectral measurement comprises a first spectral signature corresponding to mineral-associated organic material and a second spectral signature corresponding to particulate organic material, the method further comprising distinguishing at least a portion of the first spectral signature from the second spectral signature.

4. The method according to claim 1 wherein the first prediction of sequestered soil carbon comprises a predicted measure of mineral associated organic matter content in the soil.

5. The method according to claim 4 wherein the synthetic sequestered soil carbon content data comprises a measure of mineral associated organic matter content in a plurality of simulated soil samples, and the ground truth sequestered soil carbon content data comprises a measure of mineral associated organic matter content in a plurality of ground-truth soil samples.

6. The method according to claim 1 further comprising generating a second prediction of non-sequestered soil carbon content for the soil.

7. The method according to claim 6 further comprising generating a third prediction of total soil carbon content for the soil, the third prediction of total soil carbon content being based on a sum of soil carbon content of the first and second predictions.

8. The method according to claim 1 wherein the input spectral measurement comprises a high-spatial-resolution spectral measurement and a low-spatial-resolution spectral measurement.

9. The method according to claim 8 wherein the low-spatial-resolution spectral measurement comprises at least one of: satellite images and aerial images of an area of interest comprising a location of the soil.

10. The method according to claim 8 wherein the high-resolution spectral measurement comprises an at-depth spectral measurement of the soil.

11. The method according to claim 10 wherein the at-depth spectral measurement comprises a spectral measurement of a soil sample obtained at a depth of at least one of: about 0.01 m to 1 m, 0.1 m to 1 m, 0.25 m to 1 m, and 0.1 m to 10 m.

12. The method according to claim 10 wherein the at-depth spectral measurement comprises a Raman spectral measurement.

13. The method according to claim 1 wherein the synthetic training spectral measurements comprises a simulated spectral measurement generated by a physical soil simulation.

14. The method according to claim 13 wherein at least one simulated spectral measurement for a physical soil simulation comprising first and second soil components is based on a sum of a first simulated spectral measurement for the first soil component and a second simulated spectral measurement for the second soil component.

15. The method according to claim 1 wherein the synthetic training spectral measurements comprises a predicted spectral measurement generated by a generative machine learning model trained over soil properties and configured to predict spectral measurements based on soil properties.

16. The method according to claim 13 wherein the simulated spectral measurement is based on a simulation of at least one of: a vibrational frequency and a vibrational intensity of molecules represented in the physical soil simulation.

17. The method according to claim 1 wherein the first prediction comprises a measure of uncertainty.

18. The method according to claim 17 wherein the machine learning model comprises a Bayesian ensemble neural network configured to provide the measure of uncertainty for each prediction.

19. The method according to claim 1 wherein:
the ground truth sequestered soil carbon content training data corresponds to a plurality of ground truth training locations in an area of interest;
the machine learning model is configured to generate predictions of sequestered soil carbon content based on the input spectral measurement and a location identifier associated with the input spectral measurement, the machine learning model having parameters trained over a plurality of ground-truth location identifiers associated with the ground truth training spectral measurements; and
generating the first prediction of sequestered soil carbon content for the soil comprises generating the first prediction of sequestered soil carbon content for the location based on a location identifier for the soil associated with the input spectral measurement.

20. The method according to claim 1 wherein generating the first prediction of sequestered soil carbon content comprises generating an intermediate representation by a first portion of the machine learning model, combining the intermediate representation with one or more additional inputs to form a combined input, and generating the first prediction by a second portion of the machine learning model based on the combined input.
